(19) United States
(12) Patent Application Publication
(10) Pub. No.: US 2026/0086460 A1
(43) Pub. Date: Mar. 26, 2026

(54) BIFUNCTIONAL MOLECULAR GROUP STRUCTURE FOR PHOTORESIST, AND SYNTHESIS METHOD, AND USE METHOD FOR THE SAME

(71) Applicant: Shanghai Huali Integrated Circuit Corporation, Shanghai (CN)

(72) Inventors: Lingyan QIN, Shanghai (CN); Rui QIAN, Shanghai (CN)

(73) Assignee: Shanghai Huali Integrated Circuit Corporation, Shanghai (CN)

(21) Appl. No.: 19/307,729

(22) Filed: Aug. 22, 2025

(30) Foreign Application Priority Data
Sep. 25, 2024 (CN) 202411347805.3

(51) Int. Cl. G03F 7/004; C07D 487/04; G03F 7/039; G03F 7/38

(52) U.S. Cl. CPC G03F 7/0045; C07D 487/04; G03F 7/0392; G03F 7/38

(57) ABSTRACT

The present application discloses a bifunctional molecular group structure for a photoresist, including: a linker, a PAG molecule, and a PDQ molecule. The linker contains a carbon-oxygen chemical bond or a carbon-nitrogen chemical bond. Each linker is bonded to both the PAG molecule and the PDQ molecule in a number ratio, and the total number of the PAG molecule and the PDQ molecule is 3 or more. The PAG molecule is bonded to the linker through the carbon-oxygen chemical bond. The PDQ molecule is bonded to the linker through the carbon-nitrogen chemical bond. The present application also discloses a method for synthesizing the bifunctional molecular group structure for a photoresist. The present application further discloses a method for using a photoresist employing the bifunctional molecular group structure. In the present application, the ratio of the PAG molecule to the PDQ molecules can be set.

### CROSS-REFERENCES TO RELATED APPLICATIONS

This application claims priority to Chinese patent application No. CN202411347805.3, filed on Sep. 25, 2024, the disclosure of which is incorporated herein by reference in its entirety.

### TECHNICAL FIELD

The present application relates to the field of semiconductor integrated circuit manufacturing, and in particular, to a bifunctional molecular group structure for a photoresist. The present application also relates to a synthesis method for the bifunctional molecular group structure for a photoresist. The present application further relates to a method for using a photoresist employing the bifunctional molecular group structure.

### BACKGROUND

Chemically amplified photoresist components include a polymer resin, a photo acid generator (PAG), an additive, and a solvent. A conventional chemically amplified photoresist contains a base quencher, where a photo decomposable quencher (PDQ) is an basic organic molecule that decomposes upon light irradiation, thereby losing basicity thereof.

Upon light irradiation, PAG decomposes to produce acid group ions H+. During post exposure bake (PEB), H+, as a catalyst, causes cleavage of acid-labile bonds, so that the polarity of a polymer is varied, and the polymer may be dissolved in a developing solution. In addition, the generated acid further acts on the deprotection reaction of the polymer. PAG significantly increases the light sensitivity of the chemically amplified photoresist, reducing exposure energy.

Referring to FIGS. 1A to 1D, they are schematic diagrams showing mechanism of action of PAG in a photoresist in each step of an existing photolithography process; and the steps of the existing photolithography process include:
referring to FIG. 1A, a photoresist 102 is coated onto a wafer 101, the photoresist 102 containing PAG molecules 103.

Referring to FIG. 1B, exposure is performed to transfer a pattern on a mask 104 to the photoresist 102, where light 105 passes through a window area of the mask 104 to illuminate an underlying photoresist 102, thereby achieving pattern transfer. In FIG. B, PAG molecules 103 in an area of the photoresist 102 illuminated through the light 105 decompose to form a structure indicated by a mark 103a and generate acid group ions H+.

Referring to FIG. C, post exposure bake is performed. During the post exposure bake, H+, as a catalyst, causes cleavage of acid-labile bonds, so that the polarity of a polymer is varied, and the polymer may be dissolved in a developing solution.

Referring to FIG. D, an organic developing solution is sprayed for development, so that the photoresist 102 in an exposed area is removed to form a spacing pattern 102b, and a remained photoresist 102 forms a strip 102a.

For PDQ, the PDQ in the exposed area loses its basicity due to light exposure and cannot neutralize acid released by a photoacid generator, and an acid concentration is kept at a high level in the photoresist. PDQ in an unexposed area does not decompose, a high basicity is kept in the photoresist. In summary, an acid concentration difference between the exposed area and the unexposed area further increases, thereby increasing photoresist contrast.

Referring to FIGS. 2A to 2C, they are schematic diagrams showing mechanism of action of PDQ in a photoresist in each step of the existing photolithography process.

Referring to FIG. 2A, a photoresist 202 is coated onto a wafer 201, the photoresist 202 containing PDQ molecules 203.

Referring to FIG. 2B, exposure is performed to transfer a pattern on a mask to the photoresist 202. In FIG. 2B, PDQ molecules 203 in an area of the photoresist 202 irradiated by light decompose to form a structure indicated by a mark 203a, which structure loses its basicity.

Referring to FIG. 2C, post exposure bake is performed, and an organic developing solution is sprayed for development to form a pattern structure of the photoresist 202, including a spacing pattern 202b and a strip 202a.

Therefore, by adding PDQ to the photoresist, the photoresist contrast is increased since a difference in acid concentrations in the exposed area and the unexposed area can be further increased due to the feature that PDQ loses its basicity after exposure and has basicity when unexposed as well as the feature that PAG molecules produce acid after exposure.

Referring to FIG. 3, it is a distribution curve of an acid concentration and an alkaline concentration in exposed and unexposed areas after PDQ is mixed in the existing photolithography process. A curve 301 is an acid concentration curve, and a curve 302 is an alkali concentration curve. It can be seen that the acid concentration in the exposed area is significantly higher than that in the unexposed area, while the alkali concentration in the exposed area is significantly lower than that in the unexposed area. In this case, the exposed area shown by an arrow line 304 has a larger difference in the acid concentration and the alkali concentration and remains acidic. The unexposed area shown by an arrow line 305 also has a larger difference in the alkaline concentration and the acid concentration and remains basic. At the boundary between the exposed area and the unexposed area, even if some acid diffuses to the unexposed area, the acid is neutralized by the alkali in the unexposed area. Therefore, the addition of the PDQ in a photoresist improves photoresist contrast.

Referring to FIG. 4, it is a schematic diagram of mixing of PAG molecules and PDQ molecules in an existing photoresist employing mixed PAG and PDQ. The photoresist 401 contains both PAG molecules 402 and PDQ molecules 403. However, direct mixing of the PAG molecules 402 and the PDQ molecules 403 can easily result in a situation of uneven mixing. Alkali excess occurs in an area 404, and acid excess occurs in an area 405.

To improve the distribution uniformity of PAG and PDQ in the photoresist, a molecular structure exists in the related art, in which structure, PAG and PDQ are bonded together to form a dimolecular functional group. Such as structure may ensure the uniformity of PAG molecules and PDQ molecules in the photoresist, thereby guaranteeing the uniformity of the line width and roughness of the photoresist. However, at present, a bifunctional molecule exists, in which the PAG molecule and PDQ molecule are mixed in a ratio of 1:1 via a linker.

Referring to FIG. 5, it is a schematic diagram of mixing of PAG molecules and PDQ molecules in an existing photoresist employing a bifunctional molecular group in which PAG and PDQ are bonded together. In a photoresist 501, a bifunctional molecular group 504 in which PAG molecules 503 and PDQ molecules 502 are bonded together via a linker is employed, which enable uniform mixing of the PAG molecules 503 and PDQ molecules 502. However, in the related art, other PAG-to-PDQ ratios cannot be achieved.

### BRIEF SUMMARY

According to some embodiments in this application, a bifunctional molecular group structure for a photoresist disclosed in this application includes: a linker, a PAG molecule, and a PDQ molecule.

The linker contains a carbon-oxygen chemical bond or a carbon-nitrogen chemical bond.

Each linker is bonded to both the PAG molecule and the PDQ molecule in a number ratio, and the total number of the PAG molecule and the PDQ molecule is 3 or more.

The PAG molecule is bonded to the linker through the carbon-oxygen chemical bond.

The PDQ molecule is bonded to the linker through the carbon-nitrogen chemical bond.

In some cases, the linker includes a polyol ether.

In some cases, the polyol ether includes a glycerol ether or a butantetraol ether.

In some cases, a molecular structure of the glycerol ether is:

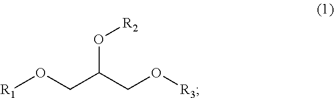

in molecular formula (1), R1, R2, and R3 represent 3 groups of the glycerol ether.

In the bifunctional molecular group structure, the PAG molecule is bonded at 1 or 2 of positions of R1, R2, and R3, and types of the PAG molecules at different positions are same or different.

The PDQ molecule is bonded at a position of R1, R2, and R3 where the PAG molecule is not bonded, and types of the PDQ molecules at different positions are same or different.

The ratio of the PAG molecule to the PDQ molecule is 1:2 or 2:1.

In some cases, a molecular structure of the butantetraol ether is:

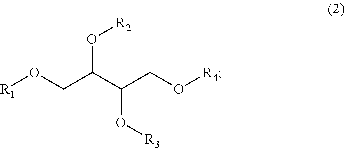

In molecular formula (2), R1, R2, R3, and R4 represent 4 groups of the butantetraol ether.

In the bifunctional molecular group structure, the PAG molecule is bonded at 1, 2, or 3 positions of R1, R2, R3, and R4, and types of PAG molecules at different positions are same or different.

The PDQ molecule is bonded at a position of R1, R2, R3, and R4 where the PAG molecule is not bonded, and types of the PDQ molecules at different positions are same or different.

The ratio of the PAG molecule to the PDQ molecule is 1:3 or 3:1.

In some cases, the PAG molecule includes di(cyclohexylsulfonyl)diazomethane or 3-hydroxy-2, 5-dioxopyrrole-1-trifluoromethyl sulfonate.

A molecular structure of di(cyclohexylsulfonyl)diazomethane is:

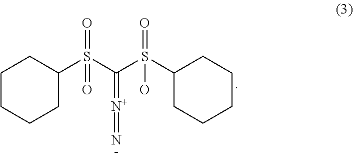

In some cases, the PDQ molecule is employed as 1,3-dioxopyrrole-3,4-tetrahydropyrrole-1-trifluoromethyl sulfonate, with a molecular structure:

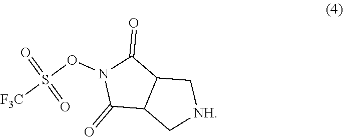 [SMILES: O=C1C2CNCC2C(=O)N1OS(=O)(=O)C(F)(F)F]

In some cases, the photoresist includes the bifunctional molecular group structure, a polymer resin, an additive, and a solvent.

According to some embodiments in this application, in a method for synthesizing the bifunctional molecular group structure for a photoresist provided by the present application, the carbon-oxygen chemical bond and the carbon-nitrogen chemical bond are obtained through a substitution reaction.

According to some embodiments in this application, a method for using a photoresist employing the bifunctional molecular group structure disclosed in this application, comprising the following steps:
Coat the photoresist on a surface of a wafer.
Perform prebake.
Perform exposure and development to form a pattern of the photoresist.
Perform post exposure bake.

In some cases, during the exposure, the PAG molecule in an exposed area releases H+ and forms acid, and the PDQ molecule loses basicity.

In some cases, during the post exposure bake, the photoresist in the exposed area decomposes under the catalysis of acid to generate new acid; and the PDQ molecule in an unexposed area neutralizes acid diffused to the unexposed area.

In some cases, when the contrast of the pattern of the photoresist is below a required value, the contrast of the pattern of the photoresist is improved by reducing the ratio of the PAG molecule to the PDQ molecule and setting the ratio of the PAG molecule to the PDQ molecule to be less than 1.

When the sensitivity of the photoresist during the exposure process is below a required value, the sensitivity of the photoresist is improved by increasing the ratio of the PAG molecule to the PDQ molecules and setting the ratio of the PAG molecule to the PDQ molecule to be greater than 1.

The present application contains a linker, such as the polyol ether, with easily bonded carbon-oxygen or carbon-nitrogen chemical bonds, facilitating to bond a plurality of PAG molecules and PDQ molecules on one linker and set the ratio of the PAG molecules to the PDQ molecules as needed. This enables synthesis of a bifunctional molecular structure that meets various situations of ratios and types of PAG and PDQ, so that a diversity degree of additives in a photoresist is increased, and thus, uniformity of PAG and PDQ in a photoresist can be ensured, and uniformity of a line width and roughness of the photoresist can be ensured while meeting users' varying requirements for the contrast and light sensitivity of a chemically amplified photoresist.

### BRIEF DESCRIPTION OF THE DRAWINGS

The utility model is further described below with reference to the accompanying drawings and detailed description:

FIGS. 1A-1D are schematic diagrams showing mechanism of action of PAG in a photoresist in each step of an existing photolithography process.

FIGS. 2A-2C are schematic diagrams showing mechanism of action of PDQ in a photoresist in each step of the existing photolithography process.

FIG. 3 is a distribution curve of an acid concentration and an alkaline concentration in exposed and unexposed areas after PDQ is mixed in the existing photolithography process.

FIG. 4 is a schematic diagram of mixing of PAG molecules and PDQ molecules in an existing photoresist employing mixed PAG and PDQ.

FIG. 5 is a schematic diagram of mixing of PAG molecules and PDQ molecules in an existing photoresist employing a bifunctional molecular group in which PAG and PDQ are bonded together.

FIG. 6A is a schematic diagram of one structure in a photoresist of the bifunctional molecular group structures for a photoresist in an embodiment of the present application.

FIG. 6B is a schematic diagram of another structure in a photoresist of the bifunctional molecular group structures for a photoresist in an embodiment of the present application.

FIG. 7A is a comparison diagram of line width roughness (LWR)/line edge roughness (LER) improvement effects between a pattern obtained through photolithography using an existing photoresist and a pattern obtained through photolithography using the photoresist in an embodiment of the present application.

FIG. 7B is a comparison diagram of critical dimension uniformity (CDU) improvement effects between a pattern obtained through photolithography using an existing photoresist and a pattern obtained through photolithography using the photoresist in an embodiment of the present application.

FIG. 8 is a comparison curve of dissolution rates versus exposure doses under different PDQ contents for a bifunctional molecular group structure for a photoresist in the embodiment of the present application.

FIG. 9 is a comparison curve of remaining photoresist thicknesses versus exposure doses under different PAG contents for a bifunctional molecular group structure for a photoresist in an embodiment of the present application.

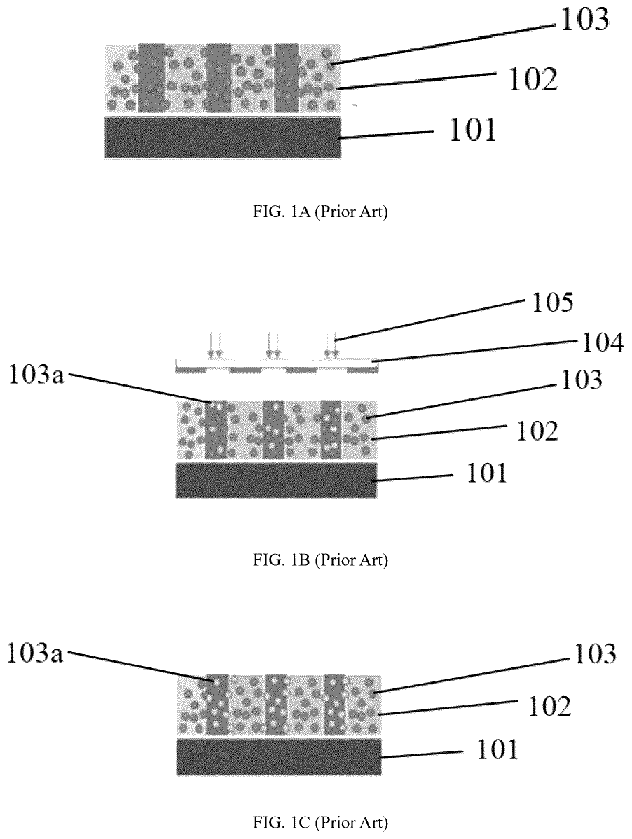

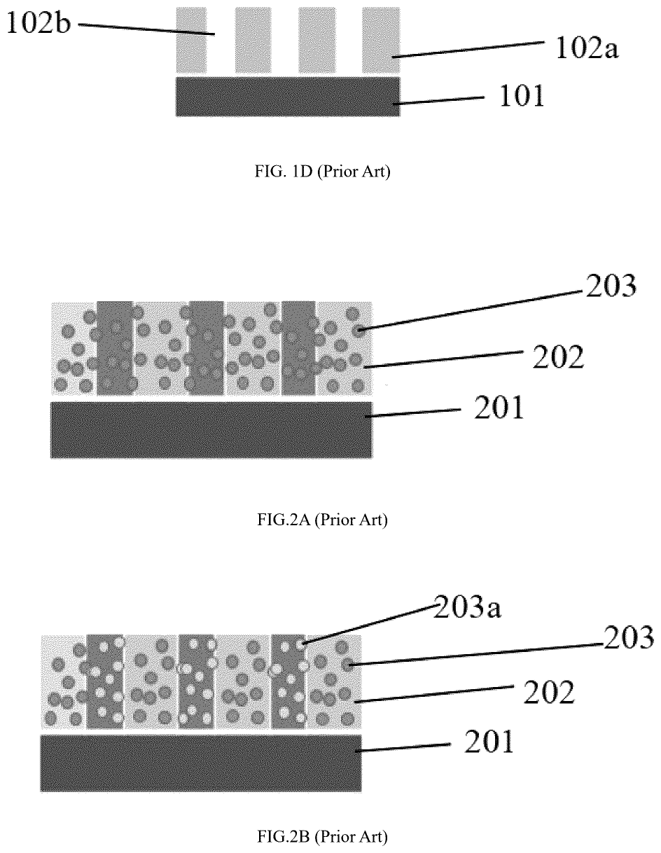

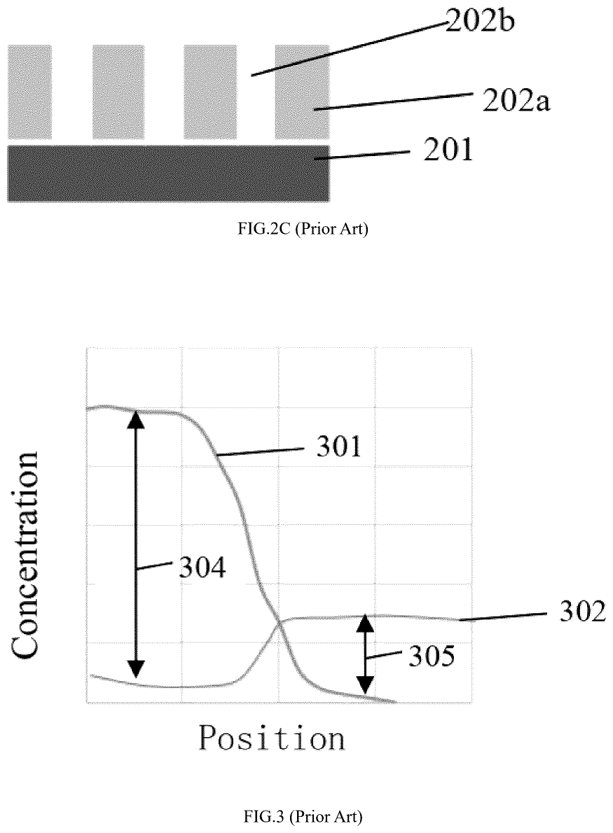

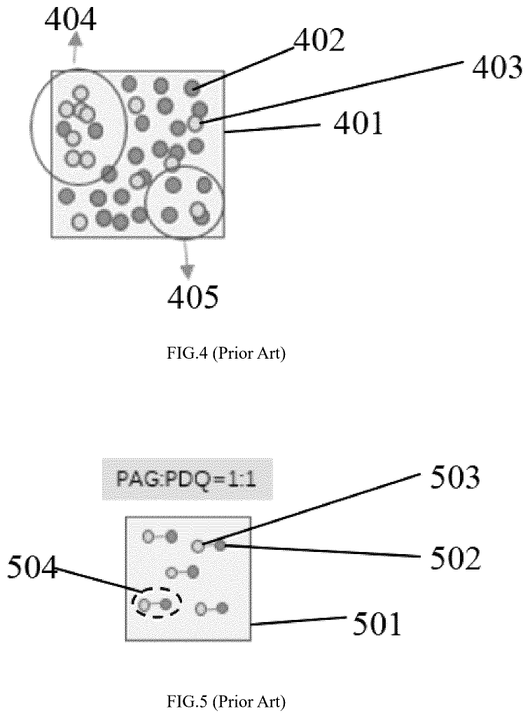

### DETAILED DESCRIPTION OF THE DISCLOSURE

The bifunctional molecular group structure for a photoresist in an embodiment of the present application includes: a linker, a PAG molecule, and a PDQ molecule.

The linker contains a carbon-oxygen chemical bond or a carbon-nitrogen chemical bond.

Each linker is bonded to both the PAG molecule and the PDQ molecule in a number ratio, and the total number of the PAG molecule and the PDQ molecule is 3 or more.

The PAG molecule is bonded to the linker through the carbon-oxygen chemical bond.

The PDQ molecule is bonded to the linker through the carbon-nitrogen chemical bond.

In the embodiment of the present application, the linker includes a polyol ether.

The polyol ether includes a glycerol ether or a butantetraol ether.

In some embodiments, the linker is a glycerol ether, and a molecular structure of the glycerol ether is:

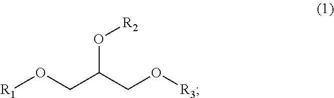

in molecular formula (1), R1, R2, and R3 represent 3 groups of the glycerol ether.

In the bifunctional molecular group structure, the PAG molecule is bonded at 1 or 2 of positions of R1, R2, and R3, and types of the PAG molecules at different positions are same or different.

The PDQ molecule is bonded at a position of R1, R2, and R3 where the PAG molecule is not bonded, and types of the PDQ molecules at different positions are same or different.

It can be seen from molecular formula (1), through replacement of these 3 groups, R1, R2, and R3, PDQ molecules or PAG molecules having a total number of 3 can be synthesized; and based on different ratios, for example, an obtained ratio of the molecular number of the PAG molecules to the molecular number of the PDQ molecules includes 1:2 or 2:1.

When the ratio of the molecular number of the PAG molecules to the molecular number of the PDQ molecules is 1:2, the bifunctional molecular group structure can be further divided into the following two types based on the different positions of the PAG molecules and the PDQ molecules due to the symmetrical positions of R1 and R3:

A first type is: 1, 3-di-PDQ-2-PAG, with a corresponding molecular structure:

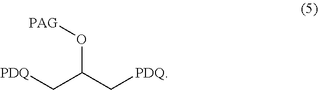

From molecular formula (5), it can be seen that the PAG molecule replaces a group corresponding to R2, and the PAG molecule and the linker are connected through a carbon-oxygen chemical bond; and the PDQ molecule replaces two groups corresponding to R1 and R3, and the PDQ molecule and the linker are connected through a carbon-nitrogen chemical bond.

A second type is: 1, 2-di-PDQ-3-PAG, with a corresponding molecular structure:

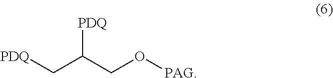

When the ratio of the molecular number of the PAG molecules to the molecular number of the PDQ molecules is 2:1, the bifunctional molecular group structure can be further divided into the following two types based on the different positions of the PAG molecules and the PDQ molecules due to the symmetrical positions of R1 and R3:

A first type is: 1, 3-di PAG-2-PDQ, with a corresponding molecular structure:

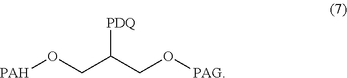

A second type is: 1, 2-di PAG-3-PDQ, with a corresponding molecular structure:

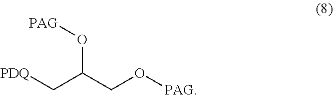

In some embodiments, the linker is butantetraol ether, with a molecular structure of the butantetraol ether:

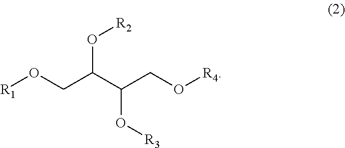

In molecular formula (2), R1, R2, R3, and R4 represent 4 groups of the butantetraol ether.

In the bifunctional molecular group structure, the PAG molecule is bonded at 1, 2, or 3 positions of R1, R2, R3, and R4, and types of PAG molecules at different positions are same or different.

The PDQ molecule is bonded at a position of R1, R2, R3, and R4 where the PAG molecule is not bonded, and types of the PDQ molecules at different positions are same or different.

The ratio of the PAG molecule to the PDQ molecule is 1:3 or 3:1.

In can be seen from molecular formula (2) that positions corresponding to R1 and R4 of the butantetraol ether are symmetrical, and positions corresponding to R2 and R3 are symmetrical. When the ratio of the PAG molecule to the PDQ molecule is 1:3, the bifunctional molecular group structure is divided into two types based on the different positions of the PAG molecule and the PDQ molecule. In a specifical molecular structure, based on molecular formula (2), one of R1 or R2 is replaced by the PAG molecule, and other 3 groups are replaced by the PDQ molecule. Similarly, when the ratio of the PAG molecule to the PDQ molecule is 3:1, the bifunctional molecular group structure is also divided into two types based on the different positions of the PAG molecule and the PDQ molecule.

In the embodiments of the present application, when the bifunctional molecular group structure includes 2 PAG molecules, types of the two PAG molecules are same or different. When the bifunctional molecular group structure includes 2 PDQ molecules, types of the two PDQ molecules are same or different.

In some embodiments, the PAG molecule includes di(cyclohexylsulfonyl) diazomethane or 3-hydroxy-2, 5-dioxopyrrole-1-trifluoromethyl sulfonate.

The molecular structure of di(cyclohexylsulfonyl)diazomethane is:

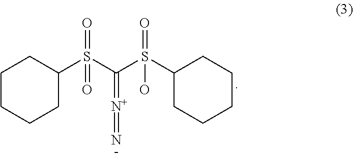

In some embodiments, the PDQ molecule is employed as 1,3-dioxopyrrole-3,4-tetrahydropyrrole-1-trifluoromethyl sulfonate, with a molecular structure:

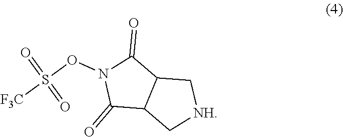 [SMILES: O=C1C2CNCC2C(=O)N1OS(=O)(=O)C(F)(F)F]

In the embodiments of the present application, a photoresist includes the bifunctional molecular group structure, a polymer resin, an additive, and a solvent. The bifunctional molecular group structure of the present application is employed, so that the PAG molecule and the PDQ molecule in the photoresist are uniformly distributed in a predetermined ratio.

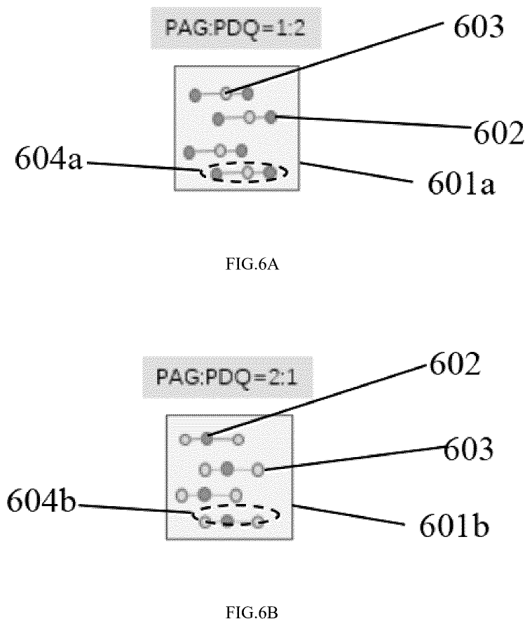

Referring to FIG. 6A, it is a schematic diagram of one structure in a photoresist of the bifunctional molecular group structures for a photoresist in an embodiment of the present application. A photoresist 601a employs a bifunctional molecular group structure 604a with a ratio of the PAG molecule to the PDQ molecule of 1:2. As can be seen, one bifunctional molecular group structure 604a includes 1 PAG molecule 603 and 2 PDQ molecules 602.

Compared with the existing bifunctional molecular group structure shown in FIG. 5 where the ratio of the PAG molecule to the PDQ molecule is 1:1, the embodiment of the present application enables the ratio of the PAG molecule to the PDQ molecule of 1:2, achieving an adjusted ratio of the PAG molecule to the PDQ molecule.

Referring to FIG. 6B, it is a schematic diagram of another structure in a photoresist of the bifunctional molecular group structures for a photoresist in an embodiment of the present application. A photoresist 601b employs a bifunctional molecular group structure 604b with a ratio of the PAG molecule to the PDQ molecule of 2:1. As can be seen, one bifunctional molecular group structure 604b includes 2 PAG molecules 603 and 1 PDQ molecule 602.

The embodiments of the present application contain a linker, such as the polyol ether, with an easily bonded carbon-oxygen or carbon-nitrogen chemical bond, facilitating to bond a plurality of PAG molecules and PDQ molecules on one linker and set the ratio of the PAG molecules to the PDQ molecules as needed. This enables synthesis of a bifunctional molecular structure that meets various situations of ratios and types of PAG and PDQ, so that a diversity degree of additives in a photoresist is increased, and thus, uniformity of PAG and PDQ in a photoresist can be ensured, and uniformity of a line width and roughness of the photoresist can be ensured while meeting users' varying requirements for the contrast and light sensitivity of a chemically amplified photoresist.

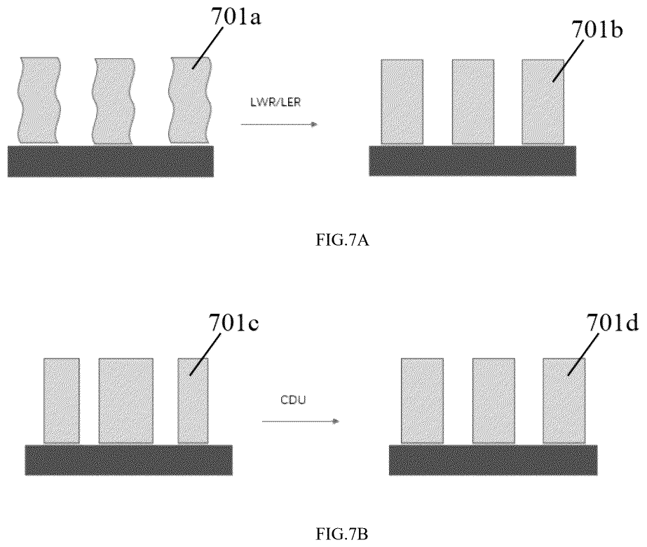

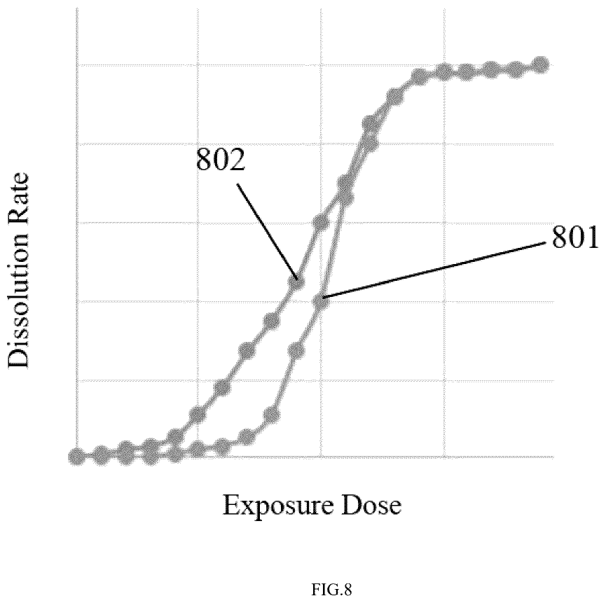

Referring to FIG. 7A, it is a comparison diagram of LWR/LER improvement effects between a pattern obtained through photolithography using an existing photoresist and a pattern obtained through photolithography using the photoresist in an embodiment of the present application. In FIG. 7A, a pattern 701a is a pattern obtained through photolithography using an existing photoresist, and a pattern 701b is a pattern obtained through photolithography using the photoresist according to the present application. It can be seen that the edge of the pattern 701b is smoother, while the edge of the pattern 701b exhibit significant fluctuations. Therefore, the present application can improve LWR and LER.

Referring to FIG. 7B, it a comparison diagram of CDU improvement effects between a pattern obtained through photolithography using an existing photoresist and a pattern obtained through photolithography using the photoresist in an embodiment of the present application. In FIG. 7B, a pattern 701c is a pattern obtained through photolithography using an existing photoresist, and a pattern 701d is a pattern obtained through photolithography using the photoresist according to the present application. It can be seen that the widths of the patterns 701c vary significantly, while the widths of the patterns 701d are uniformly consistent. Therefore, the present application can improve a CDU effect.

The present application improves LWR, LER, and the CDU effect, and on that basis, the application can also conveniently adjust the ratio of the PAG molecule to the PDQ molecule, so that the ratio of the PAG molecule to the PDQ molecule can be set based on requirements for a photoresist during application, thereby improving a photolithography process.

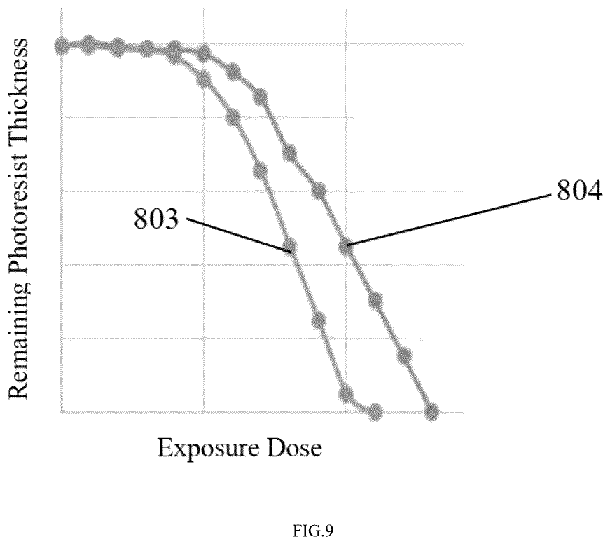

Referring to FIG. 8, it is a comparison curve of dissolution rates versus exposure doses under different PDQ contents for a bifunctional molecular group structure for a photoresist in the embodiment of the present application. In FIG. 8, a curve 801 corresponds to a curve of a dissolution rate of a photoresist with exposure doses under a higher PDQ content. A PDQ content corresponding to a curve 802 is less than a PDQ content corresponding to the curve 801. It can be seen that the curve 801 is steeper than the curve 802. Therefore, as the PDQ content increases, the dissolution rate of the photoresist becomes more sensitive to changes in exposure doses. Thus, if the sensitivity of the photoresist's dissolution rate to changes in exposure doses needs to be increased, it is only needed to increase the PDQ content. The ratio of the PAG molecule to the PDQ molecule in the bifunctional molecular group structure can be adjusted based a requirement for the PDQ content.

Referring to FIG. 9, it is a comparison curve of remaining photoresist thicknesses versus exposure doses under different PAG contents for a bifunctional molecular group structure for a photoresist in an embodiment of the present application. In FIG. 9, a curve 803 corresponds to a curve of a remaining photoresist thickness with exposure doses for a photoresist having a higher PAG content. A PAG content corresponding to a curve 804 is less than a PDQ content corresponding to a curve 803. It can be seen that the curve 803 is steeper than the curve 803. Therefore, as the PAG content increases, the remaining photoresist thickness of the photoresist becomes more sensitive to changes in exposure doses. Therefore, if the photosensitivity of the photoresist needs to be increased, it is only needed to increase the PAG content. The ratio of the PAG molecule to the PDQ molecule in the bifunctional molecular group structure can be set based a requirement for the PAG content.

The present application employs a linker composed of polyol ethers such as glycerol ether and butantetraol ether to connect PAG and PDQ, synthesizing a bifunctional molecular group structure that meets situations of various ratios and types of PAG and PDQ, thereby increasing diversity of additives.

The linker in the embodiments of the present application can bond a plurality of or various PAG and PDQ molecules in a specified ratio to one molecule, and thus, uniformity of PAG and PDQ in a photoresist can be ensured, and uniformity of a line width and roughness of the photoresist can be ensured while meeting users' varying requirements for the contrast and light sensitivity of a chemically amplified photoresist.

In an existing linker, PAG and PDQ are mixed in a ratio of 1:1. The linker in the embodiments of the present application can bond a plurality of or various PAG and PDQ molecules in a specified ratio to one molecule, thereby meeting users' different requirements for photoresist properties such as contrast, stability, and photosensitivity, for example:
1. A bifunctional molecular group with PAG: PDQ<1 increases an ammonia concentration in an unexposed area, so that a part of acid is continuously neutralized, significantly reducing an acid diffusion length, and increasing photoresist contrast.
2. A bifunctional molecular groups with PAG: PDQ>1 increases an acid concentration in an exposed areas, so that an acid diffusion length is significantly increased, enhancing photoresist sensitivity. That can be described by a contrast curve.
3. Good PAG can achieve an optimal balance among solubility, stability, sensitivity, and a process window. One type of PAG may not enable all qualified indexes. The embodiments of the present application can achieve that different photoacids are added based on a desired improving trend of a parameter.

The molecular structure of the linker in the embodiments of the present application contains chemical bonds that are easily bonded, such as carbon-oxygen bonds, and carbon-nitrogen bonds. The steric hindrance within the molecular structure of the linker should be minimal, and a plurality of functional groups are contained, which facilitates bonding of a plurality of PAG and PDQ molecules and the stability of the bifunctional molecular group.

In a method for synthesizing the bifunctional molecular group structure for a photoresist provided by the present application, the carbon-oxygen chemical bond and the carbon-nitrogen chemical bond are obtained through a substitution reaction.

In an embodiment of the present application, a method for using a photoresist employing the bifunctional molecular group structure includes the following steps:
Coat a photoresist on a surface of a wafer.
Perform prebake.

Perform exposure and development to form a pattern of the photoresist. During the exposure, the PAG molecule in an exposed area releases H+ and forms acid, and the PDQ molecule loses basicity.

Perform post exposure bake. The post exposure bake eliminates the standing wave effect for photoresist, resulting in better morphology of the photoresist.

During the post exposure bake, the photoresist in the exposed area decomposes under the catalysis of acid to generate new acid; and the PDQ molecule in an unexposed area neutralizes acid diffused to the unexposed area.

When the contrast of the pattern of the photoresist is below a required value, the contrast of the pattern of the photoresist is improved by reducing the ratio of the PAG molecule to the PDQ molecule and setting the ratio of the PAG molecule to the PDQ molecule to be less than 1.

When the sensitivity of the photoresist during the exposure process is below a required value, the sensitivity of the photoresist is improved by increasing the ratio of the PAG molecule to the PDQ molecules and setting the ratio of the PAG molecule to the PDQ molecule to be greater than 1.

The present application is described above in detail through specific embodiments. But these are not intended to limit the scope of the application. Without departing from the principle of the present application, those skilled in the art may make various modifications and improvements, which should also be considered within the scope of the present application.

What is claimed is:

1. A bifunctional molecular group structure for a photoresist, wherein the bifunctional molecular group structure comprises: a linker, a PAG molecule, and a PDQ molecule; the linker contains a carbon-oxygen chemical bond or a carbon-nitrogen chemical bond; each linker is bonded to both the PAG molecule and the PDQ molecule in a number ratio, and the total number of the PAG molecule and the PDQ molecule is 3 or more; the PAG molecule is bonded to the linker through the carbon-oxygen chemical bond; and
the PDQ molecule is bonded to the linker through the carbon-nitrogen chemical bond.

2. The bifunctional molecular group structure for a photoresist according to claim 1, wherein the linker comprises a polyol ether.

3. The bifunctional molecular group structure for a photoresist according to claim 2, wherein the polyol ether comprises a glycerol ether or a butantetraol ether.

4. The bifunctional molecular group structure for a photoresist according to claim 3, wherein a molecular structure of the glycerol ether is: 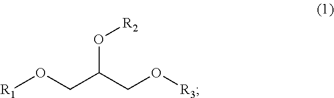
in molecular formula (1), R1, R2, and R3 represent 3 groups of the glycerol ether;
in the bifunctional molecular group structure, the PAG molecule is bonded at 1 or 2 of positions of R1, R2, and R3, and types of the PAG molecules at different positions are same or different;
the PDQ molecule is bonded at a position of R1, R2, and R3 where the PAG molecule is not bonded, and types of the PDQ molecules at different positions are same or different; and
the ratio of the PAG molecule to the PDQ molecule is 1:2 or 2:1.

5. The bifunctional molecular group structure for a photoresist according to claim 3, wherein a molecular structure of the butantetraol ether is: 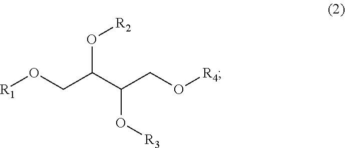
in molecular formula (2), R1, R2, R3, and R4 represent 4 groups of the butantetraol ether;
in the bifunctional molecular group structure, the PAG molecule is bonded at 1, 2, or 3 positions of R1, R2, R3, and R4, and types of PAG molecules at different positions are same or different;
the PDQ molecule is bonded at a position of R1, R2, R3, and R4 where the PAG molecule is not bonded, and types of the PDQ molecules at different positions are same or different; and
the ratio of the PAG molecule to the PDQ molecule is 1:3 or 3:1.

6. The bifunctional molecular group structure for a photoresist according to claim 1, wherein the PAG molecule comprises di(cyclohexylsulfonyl)diazomethane or 3-hydroxy-2, 5-dioxopyrrole-1-trifluoromethyl sulfonate;
a molecular structure of di(cyclohexylsulfonyl)diazomethane is:

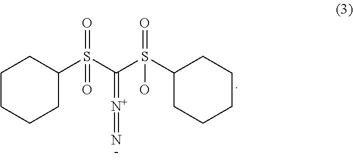

7. The bifunctional molecular group structure for a photoresist according to claim 1, wherein the PDQ molecule is employed as 1,3-dioxopyrrole-3,4-tetrahydropyrrole-1-trifluoromethyl sulfonate, with a molecular structure:

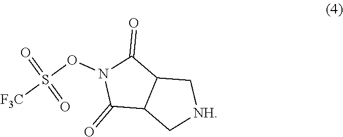 [SMILES: O=C1C2CNCC2C(=O)N1OS(=O)(=O)C(F)(F)F]

8. The bifunctional molecular group structure for a photoresist according to claim 1, wherein the photoresist comprises the bifunctional molecular group structure, a polymer resin, an additive, and a solvent.

9. A method for synthesizing the bifunctional molecular group structure for a photoresist according to claim 1, wherein the carbon-oxygen chemical bond and the carbon-nitrogen chemical bond are obtained through a substitution reaction.

10. A method for using a photoresist employing the bifunctional molecular group structure for a photoresist according to claim 1, comprising the following steps:
coating the photoresist on a surface of a wafer,
performing prebake;
performing exposure and development to form a pattern of the photoresist; and
performing post exposure bake.

11. The method for using a photoresist employing the bifunctional molecular group structure according to claim 10, wherein during the exposure, the PAG molecule in an exposed area releases H+ and forms acid, and the PDQ molecule loses basicity.

12. The method for using a photoresist employing the bifunctional molecular group structure according to claim 11, wherein during the post exposure bake, the photoresist in the exposed area decomposes under the catalysis of acid to generate new acid; and the PDQ molecule in an unexposed area neutralizes acid diffused to the unexposed area.

13. The method for using a photoresist employing the bifunctional molecular group structure according to claim 12, wherein
when the contrast of the pattern of the photoresist is below a required value, the contrast of the pattern of the photoresist is improved by reducing the ratio of the PAG molecule to the PDQ molecule and setting the ratio of the PAG molecule to the PDQ molecule to be less than 1; and
when the sensitivity of the photoresist during the exposure process is below a required value, the sensitivity of the photoresist is improved by increasing the ratio of the PAG molecule to the PDQ molecules and setting the ratio of the PAG molecule to the PDQ molecule to be greater than 1.
